(19) United States
(12) Patent Application Publication
(10) Pub. No.: US 2025/0361561 A1
(43) Pub. Date: Nov. 27, 2025

(54) METHOD FOR THE ACCURATE DETERMINATION OF AGE FROM NUCLEAR DNA BY IDENTIFYING N6-METHYLADENINE LEVELS AT SPECIFIC GENOMIC REGIONS

(71) Applicant: VELLAB BIOTECH KFT., Szeged (HU)

(72) Inventor: Tibor VELLAI, Budapest (HU)

(73) Assignee: VELLAB BIOTECH KFT., Szeged (HU)

(21) Appl. No.: 18/872,934

(22) PCT Filed: Sep. 20, 2022
(86) PCT No.: PCT/HU2022/000016

§ 371 Date: Dec. 9, 2024

(30) Foreign Application Priority Data
Jun. 13, 2022 (HU) P2200207

(51) Int. Cl. C12Q 1/6883

(52) U.S. Cl. CPC C12Q 1/6883; C12Q 2600/154

(57) ABSTRACT

A molecular biology method, by which relative (normalized to an internal control) N6-methyladenine (6mA) level at a selected specific genomic site in a tissue sample containing numerous individual cellular genomes is accurately determined, and this level is projected to a reference “6mA level-age” curve determined earlier. The relative 6mA level of the examined individual assigns her/his (biological) age on the curve. The reference curve was previously established by determining 6mA levels of a large (>1000) number of healthy individuals with known age. On the reference curve, the relative 6mA level correlates with age; the higher the 6mA level at the tested genomic site, the higher the age of the individual analyzed. The accuracy of the measurement depends on the accuracy of the method by which 6mA level is determined.

The subject of the present invention is a molecular biology method, by which the relative (normalized to an internal control) N6-methyladenine (6mA) level at a selected specific genomic site in a tissue sample containing numerous individual cellular genomes is accurately determined, and this level is projected to a reference “relative 6mA level-age” curve determined earlier. The clarified 6mA level of the examined individual with unknown identity assigns her/his (biological) age on the curve. The reference curve was previously established by determining 6mA levels of a large (>1000) number of healthy individuals with known age. On the reference curve, relative 6mA levels correlate with age; the higher the 6mA level at the tested genomic site, the higher the age of the individual examined. The accuracy of the measurement depends on the accuracy of the method by which 6mA level is determined. Until now, there is only a single method by which relative 6mA levels at selected genomic sites can be accurately determined (patent applications entitled “A PCR-based method for the accurate determination of . . . ”, with file numbers P2100409 and W2200015). During this method, individual genomes isolated from an examined tissue sample are enzymatically digested with a 6mA-dependent restriction endonuclease, the resulting genomic fragments are then ligated to a linker DNA (deoxyribonucleic acid) fragment, and finally a sequence-specific PCR-(polymerase chain reaction) based DNA amplification of the digested (target) site is achieved by using a forward primer that is simultaneously specific to the downstream part of the linker DNA fragment and the target genomic site adjacent to the linker. Ligation of the linker makes possible the direct amplification of the selected methylated (digested) adenine nucleobase (target site). The quantity of the PCR product is proportional with the relative 6mA level determined at the given genomic position.

Specifically, the innovation is a molecular biology method, by which the relative 6mA level at a specific genomic site in a tissue sample is accurately determined, and this level is then projected to a reference “relative 6mA level-age” curve established earlier in order to accurately determine the age of the individual examined. The method is also suitable for predicting the expected lifespan of an individual with known age (how long the individual will live for). The invention relies on our recent biological finding that 6mA levels at specific genomic regions correlate with (is directly proportional to) the age of the individual examined. Thus, the epigenetic process N6-adenine methylation continuously occurs in these specific genomic regions during the adult lifespan, thereby serving as a solid signature of aging rate and organismal age. Such specific nuclear regions involve active (mobile) transposable elements, also called mobile genetic elements or “jumping genes”, In the human genome, for example, 6mA levels at LINE1 (L1) sequences serve as a remarkable marker for age determination. The method can be applied in the following major areas:
1. Age determination in forensic proceedings (age can be accurately determined from the biological trace—i.e., a tissue sample left behind—of a perpetrator with unknown identity, which can significantly narrow down the number of the suspected individuals, thereby promoting the success of the investigation process).
2. Expected lifespan prediction (lifespan can be predicted in an individual with known age, and in the light of this data significant changes can be established in lifestyle and in administrative—e.g., devise—issues).
3. Identifying early stages of neurodegenerative processes (the rate of the aging process differs between normal individuals and patients affected by a neurodegenerative pathology, so when the age extrapolated by the 6mA level determined significantly differs from the chronological age of an otherwise normal looking individual, this can indicate an early—before the manifestation of cognitive decline—stage of a neurodegenerative process, and this early detection can promote the selection of a potent therapy).

### STATE OF THE TECHNIQUE

### Genetics of the Aging Process

Aging is a gradual decline in the fitness and general physiology of an organism over time which is driven by the lifelong, progressive accumulation of cellular damage including mainly misfolded, oxidized and aggregated—i.e., non-functional—proteins (Kirkwood, 2008). Such components interfere with cellular processes, thereby causing senescence (a decline in cellular functions) and, eventually, loss of the affected cell. When cell loss occurs in a large quantity, the process can lead to a tissue/organ dysfunction, which can manifest in the development of an age-associated degenerative pathology, such as cancer, various neurodegenerative diseases, diabetes, tissue atrophy and fibrosis, and immune deficiency. Such a (fatal) pathology can eventually cause the death of the organism. Despite its medical, social and economic significance, the mechanism (the primary genetic basis) of the aging process, that is the factors that generate cellular damage, remains largely unresolved (Kenyon, 2010).

The rate at which cells age is influenced by several environmental factors such as temperature, atmospheric oxygen level and nutrient availability, as well as numerous intrinsic regulatory pathways and proteins (Kenyon, 2010). Such longevity pathways include, for example, insulin/IGF1 (insulin-like growth factor) signaling (Kenyon et al., 1993), the TOR (target of rapamycin) kinase-mediated molecular cascade (Vellai et al., 2003) and the mitochondrial respiratory system (Dillin et al., 2002). Among the known lifespan-determining regulatory proteins, it is worth mentioning p53 tumor suppressor and FoxA transcription factor (Kenyon, 2010). However, these regulatory factors can only influence the rate of the aging process rather than serving as its underlying basis; although mutational or pharmacological inactivation of such a protein/pathway can extend lifespan, but the organism affected still ages and eventually dies. Autophagy (cellular self-eating), which is the main self-degradation process of eukaryotic cells, has a central role in aging control; molecular damages causing aging can be primarily broken down (eliminated) by the autophagic process (Vellai, 2009). This way cellular constituents can be rejuvenated to maintain the homeostasis (a stable functioning) of cells.

Genomic instability has been emerged as a cellular feature that is characteristic to essentially all aging cells (López-Otín et al., 2013). In the light of recent findings, a main source of genomic instability is the activity of transposable elements (TEs) that constitute a major part of eukaryotic genomes (Sturm et al., 2015; 2017; Gorbunova et al., 2021). A significant portion of TEs (called mobile genetic elements or “jumping genes”) is capable of moving from one part of the genome into another part, thereby causing an insertional mutation in the new location. If the acceptor genomic site contains a functional (coding or regulatory) DNA sequence, TE insertion can lead to a functional consequence (mutant phenotype). Thus, TEs are considered potent biological mutagens. Their significance is provided by the fact that a large portion of eukaryotic genomes consists of TE-like repetitive sequences. For example, more than half of the human genome is built up from TE-related sequences (Nurk et al., 2022). Indeed, pharmacological inhibition of mobile TE sequences has been recently shown to extend lifespan in Drosophila (Wood et al., 2016) and delay senescence in mammalian cells (De Cecco et al., 2019).

However, why TEs become gradually mobilized during the adult lifespan is still unknown. A solution could be an epigenetic regulation because DNA 5-cytosine methylation predominantly occurs in TE-like sequences and contributes their inactivation (Yoder et al., 1997), as well as DNA N6-adenine methylation has a primarily role in TE mobilization (Sturm et al., unpublished results). It is worth noting that in general 5-methylcytosine (5mC) epigenetic mark represses, while N6-methyladenine (6mA) epigenetic mark promotes, gene expression. The presence of 6mA was only recently discovered in animal and oragnellar genomes (Greer et al., 2015; Zhang et al., 2015; Wu et al., 2016), but, according to several novel studies, is considered as a result of methodological artifact in these genetic systems (Schiffers et al., 2017; O'Brown et al., 2019; Douvlataniotis et al., 2020).

### Techniques Demonstrating DNA Methylation (Epigenetic Mark)

The DNA methylation process is catalyzed by specific DNA methyltransferase enzymes, while demethylation of methylated adenine and cytosine nucleobases is conferred by specific DNA demethylases. DNA methylation is therefore a dynamic process, so the global 6mA and 5mC contents of the genome depend on the actual age of the organism and various environmental and physiological factors.

Several techniques have been developed to demonstrate the presence of methylated DNA nucleobases (6mA and 5mC epigenetic marks). They include single molecule real-time sequencing (SMRT-seq), bisulfite sequencing (the genome is treated by Na-bisulfite that converts cytosine nucleobases to uracile nucleobases, but 5mC remains unmodified by this compound), liquid chromatography-tandem mass spectrometry (LC-MS/MS) and labeling (hybridization) with a 6mA/5mC-specific antibody (Dahl and Guldberg, 2003; Flusberg et al., 2010; Rocha et al., 2010). These methods are rather costly (as they rely on expensive instruments and high expertise), time consuming and hardly available (e.g., SMRT-seq is provided by only a very few companies worldwide), thus their diagnostic application has not yet been established. Their largest disadvantage, however, is that they frequently generate artifacts (O'Brown et al., 2019; Schiffers et al., 2017; Douvlataniotis et al., 2020). This mainly comes from the fact that 6mA and 5mC epigenetic marks widely present in the genome of bacteria that often infect eukaryotic tissues and also in eukaryotic RNA found in cellular samples. Artifact can also be generated when the specificity of antibodies against 6mA and 5mC is insufficient, and by technological limitations of SMRT-seq. Furthermore, these techniques listed above are not capable of identifying the quantity (relative level) of a methylated nucleobase at a given genomic site in a tissue sample (genomic DNA is isolated from a large number of cells), rather they can only provide “yes” or “no” answer to the methylation status of a nucleobase examined.

Alternatively, 5mC levels have been tried to be determined by using 5mC-dependent/sensitive restriction endonucleases and a subsequent PCR-based amplification of the target site (Luo et al., 2016; Yao et al., 2017). These methods, however, are also unable to accurately determine methylation levels at a certain DNA site in a tissue sample. Together, these DNA methylation-analyzing technologies still remain largely unused in medical and forensic applications. A solution was provided recently by a novel method, during which genomic DNA isolated from a tissue sample is digested with a 6mA- or 5mC-specific restriction endonuclease, the resulting DNA fragments are then ligated to a linker DNA fragment, and, eventually, the target site is amplified by a PCR reaction mediated by a forward primer that is simultaneously specific to both linker sequence (10-15 nucleotides) and adjacent genomic sequence (10-15 nucleotides). The application of the linker DNA fragment makes it possible that the digested (methylated) DNA sequence is directly amplified. This is critical because the methylation ratio of a given nucleobase is a rather rare event; only a very few individual genomes in a tissue sample are methylated at a given time at at a given genomic position. Using this method, the quantity of the PCR product trustworthy reflects the relative 6mA or 5mC level of the selected genomic site (patent applications: “A PCR-based method for the accurate determination of . . . ”; file numbers: P2100409 and W2200015—the inventor gave us a permi to look at the application's content under the condition of maintaining strictly the IP rights—NDA). The more genomes are methylated at a selected genomic site among the individual ones consisting of the tissue sample examined, the higher quantity of the PCR product generated.

### An Epigenetic Clock to Determine Age

In recent years, Dr. Steve Horvath (UCLA, USA) developed an algorithm, by which the biological age of an examined tissue can be identified by determining 5mC content of certain genomic regions—Horvath's clock (Horvath, 2013). 5mC content is primarily identified by bisulfite sequencing, which is a rather costly and time-consuming procedure. Its largest disadvantage however is the inaccuracy because the method—in case of human samples—can determine biological age within 7-10 years of error means. This limitation still inhibits the application of the method in medicine and forensic genetics. Although several companies (e.g., Chronomics, Altos Labs, Zymo Research, Elysium Health és Ra Pharmaceuticals Ltds.) use this method to determine age for individuals in the diagnostic industry, they provide only an informative result for a layman procurer. The cost and time requirement of such a measurement is around 500 EUR and 1 month per sample, respectively. Moreover, it has been turned out that 5mC levels are grown at certain genomic positions, but decreased at other genomic positions, during lifespan, and that the 5-cytosine methylation process is highly affected by environmental and physiological factors. Lastly, there are organisms (e.g., the nematode Caenorhabditis elegans and fruit fly Drosophila melanogaster) that essentially lack the phenomenon of 5-cytosine methylation. In the light of these facts, one can state that identifying 5mC levels at certain genomic regions is not accurate enough to accurately determine age of an individual.

### The Problem that is Solved by the Innovation

We showed in Caenorhabditis elegans, which is a tractable genetic model for studying the regulation and mechanism of the aging process, and also in humans, that relative 6mA levels (FIG. 1) gradually increase at active TE sequences during aging (FIGS. 2 and 3). This epigenetic mark hence serves as a signature for organismal age. N6-adenine methylation presumably makes TE sequences being originally (early in life) inactive in the genome to be progressively mobile during the adult lifespan. In the C. elegans genome, we tested Tc1, Tc3 and Tc14 sequences. These are the most active TEs in this organism, and found in its genome in a relatively large copy number (e.g., Tc1 exists in 31 copies in the haploid genome). In addition, the PCR-based approach makes it possible for us to analyze the methylation status of a specific adenine nucleobase simultaneously in each copy of a TE family. We found that relative 6mA levels gradually increase with age during the adult lifespan in case of each TE family examined (FIG. 2). The present innovation is therefore based on our novel biological finding that 6mA levels progressively increase at active TE sequences as the organism ages. So, relative 6mA levels at active TE sequences serve as a solid marker for determining (biological) age from a tissue sample. According to our knowledge, such data are not available in the literature.

We also examined age-associated changes in 6mA levels at LINE1 (L1) sequences found in the human genome (L1 represents one of the most active TE families in humans). This was performed by using a primer pair that is specific to L1 sequence (amplification of a L1-specific genomic fragment was achieved by a PCR reaction). In good accordance with results obtained from C. elegans, relative (normalized to an internal control) 6mA levels gradually increased in L1 loci during aging (FIG. 3). This age-related DNA methylation process thus appears to be evolutionarily conserved. Therefore, the method presented in the current patent application is suitable for accurately determining (biological) age in various organisms ranging from worms to humans (Drosophila and dog were also tested).

Using a large number of tissue (genomic DNA) samples from human individuals with known age, a “relative 6mA level-age” curve can be generated which shows a direct correlation between the relative 6mA level at a specific genomic region and the age of individuals examined (FIG. 4). This is called the reference “relative 6mA level-age” curve. If the relative 6mA level at a specific genomic region (e.g., human L1 sequences) is identified from a tissue sample of an individual with unknown identity (age), this level (value on the Y axis) designates a single point on the reference curve. Then, extrapolating a normal line from the intersection to the X axis (ages) assigns the age of the individual tested (FIG. 4). The method is artifact-free (sequence-specific), and relatively fast and cost effective as it involves only enzymatic digestion, ligation and PCR-based DNA amplification processes, so it can be widely used as a diagnostics tool.

The method can be applied in the following major diagnostic fields:
1. Forensic genetics. In the field of criminal acts, biological trace (evidence—e.g., a hair or blood drop) from the perpetrator can often be collected. In such cases, genomic DNA is isolated from the trace left behind to determine the DNA profile of the perpetrator. The profile is frequently based on an individual DNA polymorphism (e.g., repetition number of short DNA repeats called microsatellites). Then, the result (profile) is compared with a criminal DNA database containing DNA profiles of known perpetrators, and if there is a match the identity of perpetrator can be recognized. In most cases, however, the DNA database does not contain a profile that would match with the actually identified DNA polymorphism (i.e., the profile of the perpetrator has not yet been introduced into the DNA database). In such cases, identifying relative 6mA level in a specific genomic locus from the biological trace and comparing the value with the reference “relative 6mA level-age” curve, the age of the perpetrator can be determined accurately (for example, 56±2 years—so, with a 2-year of error limit) (FIG. 4). In the light of the age determined, the number of suspects can be narrow down significantly which can largely promote the success of the investigation process.
2. Lifespan prediction in individuals with known age. Different individuals of a species live for various times. For example, if three 50 years old human individuals are chosen randomly, the first will live, for example, for 56 years, the second for 66 years while the third will die at age 84. So, lifespans of individuals with the same age can vary significantly from each other. Using the method shown above, the expected lifespan of an individual with known age can be predicted (how long she or he will live for). If the relative 6mA level at a given genomic site in a tested person is significantly higher than those expected from the reference curve at a given age, the value predicts a shorter lifespan then it is expected. So, the higher the relative 6mA level at a given age, the shorter the expected lifespan of the individual. It is important to note that this is “only” a prediction because the person may die much earlier than it is expected from her/his 6mA level, due to, for example, a fatal accident. Those getting an unfavorable prediction (a short lifespan expectancy) from the test can change their lifestyle (e.g., starting physical activity, stress-avoidance and better nutrition) significantly in order to reverse the rate at which cells age, thereby extending their lifespan. In addition, an unfavourable expectancy can also initiate a testament preparation.
3. Early detection of neurodegenerative processes. If the relative 6mA level identified in a person with known age significantly differs from those expected from the reference curve at the given age, then the value may indicate an early stage (prior to the manifestation of cognitive decline) of a neurodegenerative process (FIG. 5). This is because the rate of the aging process in a person affected by a neurodegenerative disease (e.g., Alzheimer's, Parkinson's or Huntington's disease, or ALS) differs (generally faster) from those found in non-affected persons at the same age, and this difference can be accurately identified by the method introduced in this patent application (the higher the relative 6mA level at a given chronological age, the higher the rate at which the aging process occurs). Relative 6mA levels at mobile TE sequences thus can be used as an early marker of various neurodegenerative pathologies. This is a significant finding because when a neurodegenerative disease becomes manifested (obvious cognitive problems), then major parts of the brain is already affected by neuronal demise, so pharmacological treatments applied prove to be largely ineffective (neurons that have already died cannot be revitalized). A solution can be provided by an early (well before the manifestation of cognitive decline) diagnosis, but a suitable marker for doing this has not yet been generated. When the measured relative 6mA level indicates involvement in an otherwise normal looking (healthy) individual, then it is suggested for the patient to be subjected to a further PET (positron emission tomography) or NMR (nuclear magnetic resonance) examination, and, by recognizing the presence of a degenerative process, to an immediate medical therapy.

### DETAILED DESCRIPTION OF THE INNOVATION

To determine age from DNA, the following steps have to be performed: i) the relative 6mA level at a specific genomic site in a tissue sample of a person with unknown identity is identified by a PCR-based approach (semi-quantitative PCR or real-time quantitative PCR); ii) the 6mA level identified assigns a value on a reference “relative 6mA level-age” curve; iii) extrapolating this value—intersection—to the X axis (age) assigns the age of the individual. To perform this set of experiments, the following steps are performed: i) isolation of genomic DNA from a tissue; ii) digestion of genomic DNA with a 6mA-specific restriction endonuclease (e.g., DpnI that cuts the DNA at the -GATC- sequence only when A is methylated: -GA^{Me}TC-); iii) ligation of a linker DNA fragment to the digested genomic DNA fragments; iv) PCR amplification of the target (digested) site by using a forward primer that is simultaneously specific to both linker and adjacent genomic sequence; v) quantification of the PCR product; vi) comparing the quantity of the product with that of an internal control (normalization—relative 6mA level). In this case, the PCR product trustworthily reflects the relative 6mA level at the selected genomic site. This method is described in a recent patent applications entitled “A PCR-based method for the accurate determination of . . . ”; with file number P2100409 and W2200015. The owner of this application (inventor) gave us a permit to look at the file (protocol) under condition of maintaining every aspect of the IP rights.

Using this technique, we previously identified relative 6mA levels at L1 sequences (at a given adenine nucleobase) in many hundreds of individuals with known age (identity). From data obtained, a reference “relative 6mA level-age” curve was established. The examined persons were healthy and had various ages. Then, relative 6mA level identified in a person with unknown identity was projected to the reference curve, and the intersection assigned an age on X axis (FIG. 4). Based on our measurements, deviation of data was within 2-3 years of margin of error (±2-3 years). If relative 6mA level at human L1 sequences is identified in an individual with known identity (age), and the value obtained significantly differs from that the reference curve otherwise indicates at a given age, then the difference may reflect the presence of an early stage of a neurodegenerative process (FIG. 5). In this case, the affected individual is suggested to take an imaging examination, such PET or NMR.

### A Construction Example

Method (its basis is described in the patent applications entitled “A PCR-based method for the accurate determination of . . . ”; file numbers: P2100409 and W2200015, and the inventor gave us his permit to look at the file (protocol) under condition of maintaining every aspect of IP rights—Non-Disclosure Agreement):

### Isolation of Total Genomic DNA

Isolation from blood samples was performed by using Thermo Scientific GeneJET Genomic DNA Purification Kit (#K0721).

Digestion of Genomic DNA with DpnI Restriction Endonuclease

Add the following components into an Eppendorf tube:
1 μl DpnI enzyme (10 U/μl, ThermoFisher Scientific, ER1701)
4 μl Tango Buffer (10×)
15 μl H₂O
20 μl genomic DNA solution (minimum concentration: 10 ng/μl)
(final volume: 40 μl/sample)
→incubate at 37° C. for 20 min
inactivation of DpnI: incubate samples at 80° C. for 20 min

### Ligation of Linker DNA to the Digested Genomic DNA Fragments

Add the following components to the inactivated mixture (40 μl):
4 μl T4 ligase (5 U/μl, ThermoFisher Scientific, EL0011)
5 μl ATP (2 mM) (ThermoFisher Scientific, 100 mM, R0441)
5 μl genomic linker (100×)
4 μl Tango Buffer
22 μl H₂O
final volume: 80 μl
incubate samples at 4° C. for overnight
inactivation of ligase: incubate samples at 80° C. for 20 min

DNA sequence of the linker:
5′-TAG ATC TGA CCT AAC GGT AAG AGA GTT TCA TAA
TAt ttt ttt ttt ttt ttT AT-TAT GAA ACT CTC TTA
CCG TTA GGT CAG ATC TA-3′

### PCR Reaction

6mA sample (DpnI)—Forward (left) primer: 5′-ATG AAA CTC TCT TAC CGT TAG GTC AGA TCT Atc aac-3′ and reverse (right) primer: 5′-tga acg ttg gcc tgc ctt gc-3′

### Reaction Mixture:

10 μl ABI master mix (2×-Cat. number: K0171)
3.5 μl digested/ligated DNA solution (template)
3 μl primer mix (5-5 μM)
3.5 μl H₂O
final volume: 20 μl/sample

### PCR Condition (to DpnI-Digested Samples):

1. initial denaturation: 95° C., 30 sec
2. denaturation: 95°, 10 sec
3. anealling and extension: 71° C., 30 sec
4. repeat steps 2 and 3 by 40×
5. store samples at 4° C.

Control (PvuII-digested) samples—forward (left) primer: 5′-tga atg aaa tga agc gag aag gga agt tta gag-3′ and reverse (right) primer 5′-tga acg ttg gcc tgc ctt gc-3′

### Reaction Mixture:

10 μl ABI master mix (2×)
1 μl DNA solution (template)
3 μl primer mix (5-5 μM)
6 μl H₂O
final volume: 20 μl

### PCR Condition (for PvuII-Digested Samples):

1. initial denaturation: 95° C., 60 sec
2. denaturation: 95°, 10 sec
3. anealling and extension: 60° C., 45 sec
4. repeat steps 2 and 3 by 55×
5. store samples at 4° C.

### Gel Documentation

Run DNA samples (PCR products) on 1% agarose gel, 80 mV, photo by a Kodak camera.

### Novelty of the Invention

1. Recognition that relative 6mA levels at active TE sequences (e.g., L1 in the human genome) progressively increase with age. Based on this finding, age can be accurately determined from nuclear DNA isolated from an individual with unknown identity.
2. Recognition that this process is evolutionarily conserved (in C. elegans, Tc1 sequences—the most active TE family in this organism—become progressively N6-adenine methylated during aging).
3. Recognition that relative 6mA levels identified at active TE sequences are predictive to the expected lifespan of an individual with known age.
4. Recognition that relative 6mA levels identified at active TE sequences can be indicative for the presence of a neurodegenerative process.

### REFERENCES

Dahl C, Guldberg P. DNA methylation analysis techniques. Biogerontology 4, 233-250 (2003). PMID: 14501188
De Cecco M, Ito T, Petrashen A P, Elias A E, Skvir N J, Criscione S W, Caligiana A, Brocculi G, Adney E M, Boeke J D, Le O, Beauséjour C, Ambati J, Ambati K, Simon M, Seluanov A, Gorbunova V, Slagboom P E, Helfand S L, Neretti N, Sedivy J M. L1 drives IFN in senescent cells and promotes age-associated inflammation. Nature 2019 February; 566(7742): 73-78. PMID: 30728521
Dillin A, Hsu A L, Arantes-Oliveira N, Lehrer-Graiwer J, Hsin H, Fraser A G, Kamath R S, Ahringer J, Kenyon C. Rates of behavior and aging specified by mitochondrial function during development. Science. 2002 Dec. 20; 298(5602): 2398-2401. PMID: 12471266
Douvlataniotis K, Bensberg M, Lentini A, Gylemo B, Nestor C E. No evidence for DNA N 6-methyladenine in mammals. Sci Adv. 2020 Mar. 18; 6(12): eaay3335. PMID: 32206710
Flusberg B A, Webste D R, Lee J H, Travers K J, Olivares E C, Clark T A, Korlach J, Turner S W. Direct detection of DNA methylation during single-molecule, real-time sequencing. Nat Methods 7, 461-465 (2010). PMID: 20453866
Gorbunova V, Seluanov A, Mita P, McKerrow W, Fenyö D, Boeke J D, Linker S B, Gage F H, Kreiling J A, Petrashen A P, Woodham T A, Taylor J R, Helfand S L, Sedivy J M. The role of retrotransposable elements in ageing and age-associated diseases. Nature. 2021 August; 596(7870): 43-53. PMID: 34349292
Greer E L, Blanco M A, Gu L, Sendinc E, Liu J, Aristizábal-Corrales D, Hsu C-H, Aravind L, He C, Shi Y. DNA Methylation on N6-Adenine in C. elegans. Cell 161, 868-878 (2015). PMID: 25936839
Horvath S. DNA methylation age of human tissues and cell types. Genome Biol. 2013; 14(10): R115. PMID: 24138928
Kenyon C J. The genetics of ageing. Nature. 2010 Mar. 25; 464(7288): 504-512. PMID: 20336132
Kenyon C, Chang J, Gensch E, Rudner A, Tabtiang R. A C. elegans mutant that lives twice as long as wild type. Nature. 1993 Dec. 2; 366(6454): 461-464. PMID: 8247153
Kirkwood T B. A systematic look at an old problem. Nature. 2008 Feb. 7; 451(7179): 644-647. PMID: 18256658
López-Otín C, Blasco M A, Partridge L, Serrano M, Kroemer G. The hallmarks of aging. Cell. 2013 Jun. 6; 153(6): 1194-1217. PMID: 23746838
Luo G-Z, Wang F, Weng X, Chen K, Hao Z, Yu M, Deng X, Liu J, He C. Characterization of eukaryotic DNA N(6)-methyladenine by a highly sensitive restriction enzyme-assisted sequencing. Nat. Commun. 7, 11301 (2016). PMID: 27079427
Nurk S, Koren S, Rhie A, Rautiainen M, Bzikadze A V, Mikheenko A, Vollger M R, Altemose N, Uralsky L, Gershman A, Aganezov S, Hoyt S J, Diekhans M, Logsdon G A, Alonge M, Antonarakis S E, Borchers M, Bouffard G G, Brooks S Y, Caldas G V, Chen N C, Cheng H, Chin C S, Chow W, de Lima L G, Dishuck P C, Durbin R, Dvorkina T, Fiddes I T, Formenti G, Fulton R S, Fungtammasan A, Garrison E, Grady P G S, Graves-Lindsay T A, Hall I M, Hansen N F, Hartley G A, Haukness M, Howe K, Hunkapiller M W, Jain C, Jain M, Jarvis E D, Kerpedjiev P, Kirsche M, Kolmogorov M, Korlach J, Kremitzki M, Li H, Maduro V V, Marschall T, McCartney A M, McDaniel J, Miller D E, Mullikin J C, Myers E W, Olson N D, Paten B, Peluso P, Pevzner P A, Porubsky D, Potapova T, Rogaev E I, Rosenfeld J A, Salzberg S L, Schneider V A, Sedlazeck F J, Shafin K, Shew C J, Shumate A, Sims Y, Smit A F A, Soto D C, Sović I, Storer J M, Streets A, Sullivan B A, Thibaud-Nissen F, Torrance J, Wagner J, Walenz B P, Wenger A, Wood J M D, Xiao C, Yan S M, Young A C, Zarate S, Surti U, McCoy R C, Dennis M Y, Alexandrov I A, Gerton J L, O'Neill R J, Timp W, Zook J M, Schatz M C, Eichler E E, Miga K H, Phillippy A M. The complete sequence of a human genome. Science. 2022 April; 376(6588): 44-53. PMID: 35357919
O'Brown Z K, Boulias K, Wang J, Wang S Y, O'Brown N M, Hao Z, Shibuya H, Fady P-E, Shi Y, He C, Megason S G, Liu T, Greer E L. Sources of artifact in measurements of 6mA and 4mC abundance in eukaryotic genomic DNA. BMC Genomics. 2019 Jun. 3; 20(1): 445. PMID: 31159718
Rocha M S, Castro R, Rivera I, Kok R, Smulders Y M, Jakobs C, de Almeida I T, Blom H J. Global DNA methylation: comparison of enzymatic- and non-enzymatic-based methods. Clin. Chem. Lab. Med. 48, 1793-1798 (2010). PMID: 20979561
Schiffers S, Ebert C, Rahimoff R, Kosmatchev O, Steinbacher J, Bohne A-V, Spada F, Michalakis S, Nickelsen J, Müller M, Carell T. Quantitative LC-MS Provides No Evidence for m 6 dA or m 4 dC in the Genome of Mouse Embryonic Stem Cells and Tissues. Angew Chem Int Ed Engl. 2017 Sep. 4; 56(37): 11268-11271. PMID: 28371147
Sturm Á, Ivics Z, Vellai T. The mechanism of ageing: primary role of transposable elements in genome disintegration. Cell Mol Life Sci. 2015 May; 72(10): 1839-1847. PMID: 25837999
Sturm Á, Perczel A, Ivics Z, Vellai T. The Piwi-piRNA pathway: road to immortality. Aging Cell. 2017 October; 16(5): 906-911. PMID: 28653810
Vellai T. Autophagy genes and ageing. Cell Death Differ. 2009 January; 16(1): 94-102. PMID: 19079287
Vellai T, Takacs-Vellai K, Zhang Y, Kovacs A L, Orosz L, Müller F. Genetics: influence of TOR kinase on lifespan in C. elegans. Nature. 2003 Dec. 11; 426(6967): 620. PMID: 14668850
Wood J G, Jones B C, Jiang N, Chang C, Hosier S, Wickremesinghe P, Garcia M, Hartnett D A, Burhenn L, Neretti N, Helfand S L. Chromatin-modifying genetic interventions suppress age-associated transposable element activation and extend lifespan in Drosophila. Proc. Natl. Acad. Sci. USA. 2016 Oct. 4; 113(40): 11277-11282. PMID: 27621458
Wu T P, Wang T, Seetin M G, Lai Y, Zhu S, Lin K, Liu Y, Byrum S D, Mackintosh S G, Zhong M, Tackett A, Wang G, Hon L S, Fang G, Swenberg J A, Xiao A Z. DNA methylation on N(6)-adenine in mammalian embryonic stem cells. Nature. 2016 Apr. 21; 532(7599): 329-333. PMID: 27027282
Yoder J A, Walsh C P, Bestor T H. Cytosine methylation and the ecology of intragenomic parasites. Trends Genet. 1997 August; 13(8): 335-340. PMID: 9260521
Zhang G, Huang H, Liu D, Cheng Y, Liu X, Zhang W, Yin R, Zhang D, Zhang P, Liu J, Li C, Liu B, Luo Y, Zhu Y, Zhang N, He S, He C, Wang H, Chen D. N6-methyladenine DNA modification in Drosophila. Cell 161, 893-906 (2015). PMID: 25936838
Yao B, Cheng Y, Wang Z, Li Y, Chen L, Huang L, Zhang W, Chen D, Wu H, Tang B, Jin P. DNA N6-methyladenine is dynamically regulated in the mouse brain following environmental stress. Nat. Commun. 8, 1122 (2017). PMID: 29066820

### FIGURE LEGENDS

FIG. 1. N6-methyladenine (6mA) epigenetic mark. Adenine (A) can be converted to N6-methyladenine (6mA) by the addition of a methyl group (—CH₃). The methyl group is transferred to the N atom at position 6 of adenine by a DNA N6-adenine methyltransferase enzyme. A N6-methyladenine demetilase enzyme can remove the —CH₃ group from 6mA, thereby generating an adenine. Thus, the methylation and demethylation processes together influence the methylation status of a single adenine in a genomic site. The —CH₃ group is indicated by red coloring.

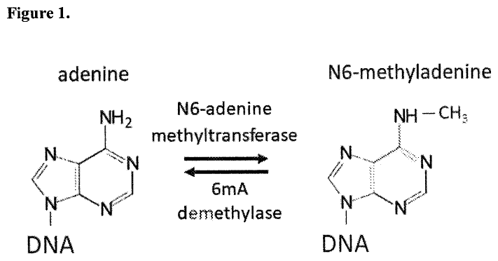

FIG. 2. In C. elegans, 6mA levels gradually increase with age in active TE loci. a) Gel photos showing relative 6mA levels at active TE sequences. The age (days) of adult animals is shown on the top. Control samples were generated by a simple PCR amplification of another genomic site. The quantities of PCR products are nearly equal in each sample (equal amounts of template DNA were used for the analysis). Tc1, Tc3 and Tc14: active transposable elements in the C. elegans genome. cep-1 (C. elegans p53 homolog): a non-TE gene (negative control). 6mA levels progressively increase at active TE sequences. a′) Quantification of PCR products. TE data were normalized to control ones. Relative 6mA levels gradually increase at active TE sequences during the adult lifespan. cep-1 gene does not show such changes. Bars indicate ±S.E.M. b) Gel photos showing 6mA levels at active TE sequences in wild-type animals (control) vs. long-lived daf-2(-) mutants (loss-of-function mutations in daf-2 can double lifespan; Kenyon et al., 1993). Relative 6mA levels at Tc1, Tc3 and Tc14 sequences progressively increase as the animal ages. b′) Quantification of data obtained from daf-2(-) mutant animals. c) Rates at which N6-adenine methylation occurs in Tc1 loci differ between wild-type and daf-2(-) mutant animals. In both strains, the maximal 6mA levels reach a similar value in old animals, but in daf-2(-) mutants living two times longer than control the rate at which 6mA levels grow is two times lower. Relative levels of 6mA at TE sequences thus depend on the actual age of the organism. c′) Although the rate at which 6mA levels in Tc3 loci change differs between wild-type (control) and daf-2(-) mutant animals, but gradually increases in both strains during lifespan. c″) Relative 6mA levels at Tc14 sequences gradually increase with age, although with a different rate, in wild-type and daf-2(-) mutant animals. Thus, relative 6mA levels at active TE sequences serve as an accurate marker of age.

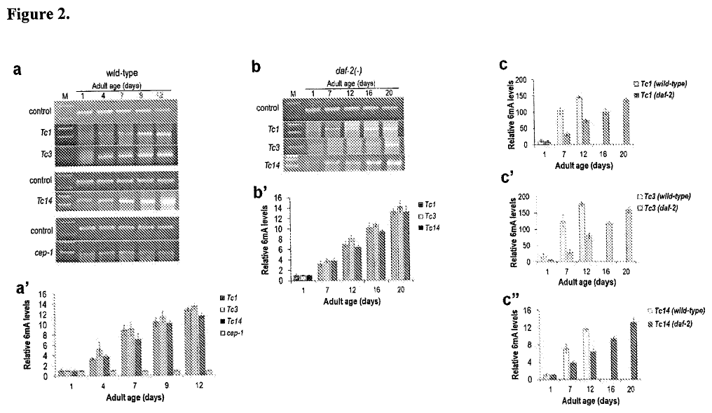

FIG. 3. Determination of N6-methyladenine (6mA) levels in human LINE1 loci in individuals with different ages. Gel photo showing that relative 6mA levels at human LINE1 sequences increase with age. Each sample was tested by two times. Up: control DNA samples (a simple PCR amplification of another genomic region). Bottom: 6mA levels from the same individuals. The nearly constant level of control samples indicates that every test contained a nearly equal amount of template DNA. Gel documentation of PCR products. M: molecular weight marker.

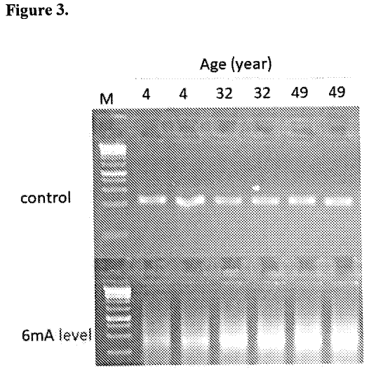

FIG. 4. Lifespan prediction in humans by identifying relative 6mA levels at TE sequences. A reference “relative 6mA level-age” curve (blue line) was previously generated by using many hundreds of healthy samples with known chronological ages. Identifying the relative 6mA level at an active TE sequence (dotted red line) in the sample of an individual with unknown identity (age), the value obtained can be projected to the reference curve in order to uncover the age (e.g., 70 years).

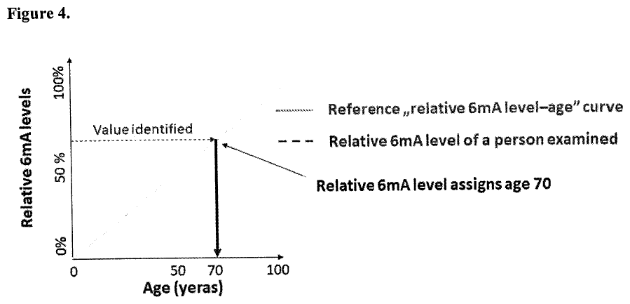

FIG. 5. Predicting early stages of neurodegenerative processes by identifying relative N6-methyladenine (6mA) levels in active TE loci. The rate of the aging process differs between healthy individuals and patients affected by a neurodegenerative disease. If the relative 6mA level (dotted red line) identified in a person with known identity (e.g., 50 years old) assigns an age (e.g., 70 years) that significantly differs from that (50 years) determined by the reference curve (green double arrows), then the value may indicate the presence of a neurodegenerative process in the individual tested.

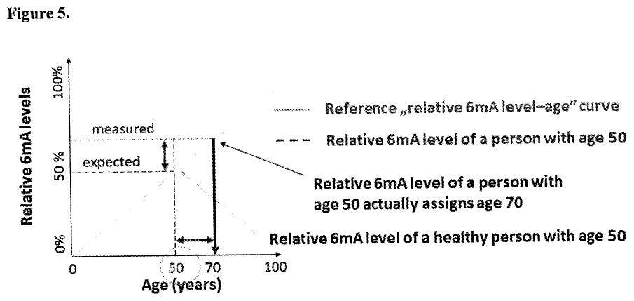

1. Method/procedure for accurately determining age from human nuclear DNA by identifying relative N6-methyladenine levels at sequences of an active transposable element family.

2. Method/procedure according to claim 1 when the organism examined is any eukaryotic species.

3. Method/procedure according to claim 1 when determination of N6-methyladenine levels is achieved by any adequate molecular technology.

4. Method/procedure according to claim 1 when the target sequence is any transposable element-like repetitive sequence, more any nuclear DNA sequence.

5. Method/procedure according to claim 1 when the type of the tissue from which genomic DNA is isolated for the analysis is arbitrary.

6. Method/procedure according to claim 1 when the determination of a reference “relative N6-methyladenine level-age” curve is achieved in any population of the organism.

7. Method/procedure according to claim 1 when the aim of the analysis is to determine the age of a person with unknown identity.

8. Method/procedure according to claim 1 when the aim of the analysis is to predict expected lifespan in an individual with known identity (age).

9. Method/procedure according to claim 1 when the aim of the analysis is to identify an early stage (before manifesting signs of cognitive dysfunctions) of a neurodegenerative process.
